(19) United States
(12) Patent Application Publication
(10) Pub. No.: US 2022/0135653 A1
(43) Pub. Date: May 5, 2022

(54) Intranasal Antiviral Therapy for Mucosal Protection Against Virus Infections

(71) Applicant: Bravado Pharmaceuticals, LLC, Lutz, FL (US)

(72) Inventors: Brian McMillan, Wesley Chapel, FL (US); Ted Koontz, Lutz, FL (US); Molly Berberich, Lutz, FL (US); Ashley Nicole Mandell, Wesley Chapel, FL (US); Garrett James Kriston, Clearwater, FL (US); Liznair Gonzalez Feliciano, Wesley Chapel, FL (US); Emma Price, Valrico, FL (US)

(21) Appl. No.: 17/502,086

(22) Filed: Oct. 15, 2021

Related U.S. Application Data
(60) Provisional application No. 63/110,256, filed on Nov. 5, 2020.

(51) Int. Cl. C07K 16/10; A61K 47/02; A61K 47/38

(52) U.S. Cl. CPC C07K 16/10; A61K 47/38; A61K 47/02

(57) ABSTRACT

A topical intranasal antiviral composition for protecting against virus infections. The composition comprises an antigen binder and a pharmaceutical suspender material to allow effective delivery into the nasal cavity. Examples of materials that could be used in the pharmaceutical suspender are microcrystalline cellulose or sodium carboxymethylcellulose (Na⋅CMC). One particular target for the antigen binder could be SARS-CoV-2. For example, the antigen binder could target the S1 subunit of SARS-CoV-2. The composition could be made by a process in which the pharmaceutical vehicle is prepared, and then the antigen binder is added to the pharmaceutical vehicle to make a bioactive mixture, and then adding solid sodium chloride to the bioactive mixture. Also disclosed are methods of protection against virus infection using the intranasal antiviral composition. For example, in the case of SARS-CoV-2, the antigen binder would block the virus particles from attaching to ACE2 receptors on host cells of the nasal mucosa or nasopharynx. This blocking action would protect against virus infection.

### TECHNICAL FIELD

This invention relates to intranasal therapy for protection against virus infections.

### BACKGROUND

Covid-19 is caused by the novel severe acute respiratory syndrome coronavirus 2 (SARS-CoV-2). SARS-CoV-2 transmission occurs via respiratory droplets and contact routes. Droplet transmission can occur through direct contact when a person is exposed to infective respiratory droplets near someone with respiratory symptoms such as coughing and sneezing. Being within close distance, people can be exposed to the droplets through the nose. Transmission can also occur through indirect contact by way of fomites on surfaces in the immediate environment around the infected person. Airborne transmission may be possible when aerosol-generating procedures are performed, such as endotracheal intubation, cardiopulmonary resuscitation, administration of nebulized medications, and others.

In the initial stages of infection, SARS-CoV-2 targets nasal and bronchial mucosal cells through the viral structural spike (S) protein that binds to the angiotensin-converting enzyme 2 (ACE2) receptor on the host cells. This binding interaction allows entry of the virus into the host cell. SARS-CoV-2 infection may be asymptomatic or it may cause a wide spectrum of symptoms, ranging from mild symptoms of upper respiratory tract infection to life-threatening sepsis. Unfortunately, the public is still awaiting a vaccine or proven effective therapy against SARS-CoV-2 infection. While many clinical trials are currently underway, the mainstay of therapy remains supportive care. Until an effective vaccine is available, the primary methods to reduce spread of SARS-CoV-2 are face masks, social distancing, and contact tracing. Thus, additional therapeutic or preventive strategies to combat the spread of SARS-CoV-2 are needed.

### SUMMARY

In one aspect, this invention is a topical intranasal antiviral composition. The composition is a liquid and may have the form of any of the various types of liquid mixtures, such as a solution, suspension, emulsion, gel, sol, liquid foam, etc. The ingredients of the nasal spray composition comprise the following.

Antigen Binder. This invention uses one or more antigen binders for binding to the virus. As used herein, ‘antigen binder’ means an antibody or antibody-like polypeptide macromolecule that have one or more antibody variable domains incorporated therein to confer binding capability against a target antigen on the virus.

In some cases, the antigen binder is an antibody, in the manner that the term is traditionally used. Examples of antibodies include IgG, IgM, IgA, IgD, and IgE. This invention also encompasses antibodies from other animal species, such as antibodies from camelids (V_{H}H), chickens (such as IgY), rodents, rabbits, etc. The antibody could also be chimeric or humanized antibodies. The antibodies could be monoclonal or polyclonal.

The term antigen binder also encompasses the various types of antigen binding fragments of antibodies. Examples of antibody fragments include Fab fragment, Fab′ fragment, F(ab)2 fragment, F(ab′)2 fragment, Fv fragment in which one variable heavy domain (V_{H}) and one variable light (V_{L}) domain are linked by noncovalent interactions, disulfide-linked Fv, single-chain Fv, and other antigen-binding fragments.

The term antigen binder also encompasses the various alternate antibody molecular formats that have antibody-like functions. Such alternate formats include diabodies, minibodies, nanobodies, single-chain Fab, and single antibody variable domain such as dAb, V_{H} (antibody heavy chains), camelid V_{H}H, or V_{L} (antibody light chains), homodimers and heterodimers of antibody heavy chains or light chains. Another antibody-type format that could be used are miniproteins. See Cao et al, “De novo design of picomolar SARS-CoV-2 miniprotein inhibitors” (2020 Oct. 23) Science; 370(6515):426-431, which is incorporated by reference herein. In some embodiments, such miniprotein may have a size of 40-90 amino acid residues.

The antigen binders used in this invention may be designed as variations or derivatives of antibodies originating from any species and produced by recombinant genetic engineering. The antigen binders used in this invention may be produced in any suitable way, such as directly from animals (e.g. polyclonal antibodies obtained from serum), B-cells, or hybridomas; or produced by recombinant genetic technology so that it could be produced from yeast, bacteria, or other types of cells.

The antigen binders could be modified versions of any of the foregoing (e.g. modified by the covalent attachment of polyethylene glycol or other suitable polymer or a humanized V_{H}H). The antigen binders could be bispecific, multispecific, bivalent, or multivalent. The antigen binders used in this invention could be used as a single type of antigen binder (e.g. single monoclonal antibody) or multiple types (two or more) of antigen binders. Examples of how multiple types of antigen binders could be used include making a cocktail mixture of different antigen binders or using polyclonal antibodies.

The intranasal antiviral composition may have any suitable concentration amount of the antigen binder. In some embodiments, the concentration of the antigen binder in the antiviral composition is 1-80 mg/ml; and in some cases, 3-40 mg/ml. In the case of multiple types of antigen binders being used (such as a cocktails thereof or polyclonal antibodies), the concentration refers to the total concentration of all the different antigen binders in aggregate.

The antigen binder used in this invention may have high binding affinity for the target antigen. One conventional way to assess binding potency is by measuring K_{D}, the equilibrium dissociation constant between the antigen binder and its antigen. The K_{D} is the ratio of the antigen binder's dissociation rate (k_{off}), how quickly it dissociates from its antigen, relative to the antigen binder's association rate (kₒₙ), how quickly it binds to its antigen. A lower K_{D} value indicates a higher affinity for the target antigen. In some embodiments, K_{D} is of the antigen binder is less than 10⁻⁵; in some cases, less than 10⁻′; and in some cases, less than 10⁻⁹. For standardization, this would be measured at a temperature of 37° C., pH of 7.0, and molar ionic strength of 0.16.

In the case of the antigen binder being multivalent, cocktail mixture, or polyclonal, or situation where a cumulative binding strength would be useful information, this cumulative binding strength could be measured by avidity. In some embodiments, with thiocyanate as the chaotropic agent, the avidity index of the antigen binder(s) is at least 50%; and in some cases, at least 70% (measured as a percentage). Techniques for measuring avidity index are described in P J Klasse, “How to assess the binding strength of antibodies elicited by vaccination against HIV and other viruses” (2016) Expert Rev Vaccines, 15(3): 295-311; and Alexander et al, “What Do Chaotrope-Based Avidity Assays for Antibodies to HIV-1 Envelope Glycoproteins Measure?” (2015) J Virol. 89(11): 5981-5995. These articles are incorporated by reference herein. For standardization, this would be measured at a temperature of 37° C., pH of 7.0, and molar ionic strength of 0.16.

Viral Antigens. The antigen binders may target any of various viruses that could be transmitted by nasal inhalation. Examples of such viruses include coronavirus, influenza virus, and rhinovirus. By binding to the virus, this would work to neutralize the virus (e.g. by blocking entry into the host cells). In some embodiments, the target virus is a coronavirus. Examples of particular coronaviruses that could be targeted include those identified as MERS-CoV, SARS-CoV, and SARS-CoV-2. These coronaviruses have three relevant proteins that could be targeted by the antigen binder. These are the membrane (M) protein, envelope (E) protein, and spike (S) protein that are anchored in the viral envelope. In some embodiments, the target antigen is the spike (S) protein of the coronavirus particle.

More specifically, the target antigen could be the S1 subunit or the S2 subunit of the spike protein, which play a key role in host cell invasion. More particularly, the target antigen could be the receptor-binding domain (RBD) of the S1 subunit of the spike protein. The RBD is the region that recognizes and binds to the host angiotensin-converting enzyme 2 (ACE2). The RBD region consists of residues number 319-541 of the S1 subunit. More information about potential target antigens are described in Huang et al, “Structural and functional properties of SARS-CoV-2 spike protein: potential antivirus drug development for COVID-19” (2020, Aug. 3) Acta Pharmacologica Sinica, 41:1141-1149; and Sternberga et al, “Structural features of coronavirus SARS-CoV-2 spike protein: Targets for vaccination” (2020, Sep. 15) Life Sci, 257:118056. These articles are incorporated by reference herein.

In some embodiments, the target virus is a rhinovirus. Examples of target antigens for rhinovirus include its capsid proteins: VP1, VP2, VP3, and VP4. In some embodiments, the target virus is influenza A virus. Examples of target antigens for influenza A virus include its viral envelope proteins, hemagglutinin (H) and neuraminidase (N).

Pharmaceutical Suspender. The intranasal antiviral composition further comprises a pharmaceutical suspender material to give the composition physical properties (e.g. viscosity, flowability, homogeneity) to allow effective application into the nasal cavity and deposition onto the nasal or nasopharynx mucosa. The pharmaceutical suspender comprises one or more suspending agents. Examples of suspending agent that could be used include microcrystalline cellulose (MCC), sodium carboxymethylcellulose (Na⋅CMC), and polyvinylpyrrolidone (PVP).

Any suitable amount of the pharmaceutical suspender may be used to achieve the desired properties for the nasal spray composition. In some embodiments, the intranasal antiviral composition contains pharmaceutical suspender at a concentration of 0.25-5 w/v %; and in some cases, 0.5-4 w/v %. In cases where the pharmaceutical suspender comprises multiple (two or more) different suspending agents, this concentration refers to the aggregate amount representing all the suspending agents.

In some embodiments, the composition comprises 0.05-0.75 grams Na⋅CMC per 100 ml; and in some cases 0.05-0.45 grams Na⋅CMC per 100 ml. Expressed as mass fraction (weight %), in some embodiments, the composition comprises 0.04-0.70 wt % Na⋅CMC; and in some cases, 0.04-0.40 wt %. In some embodiments, the composition comprises MCC at an amount that is 4-12 times the amount of Na⋅CMC by weight; and in some cases, 6-10 times the amount.

Permeation Enhancers, Absence Thereof. As explained below, it may be desirable to have local action only and avoid systemic absorption. Avoiding the use of penetration enhancers may be effective in preventing or reducing unwanted systemic absorption. As such, in some embodiments, the antiviral composition may omit any conventional penetration enhancers (also referred to as absorption or permeation enhancers), such as oleic acid and other lipids, cyclodextrins, chitosan, bile salts, fatty acids and derivatives (e.g. palmitic acid, palmitoleic acid, stearic acid, oleyl alcohol, oleic acid, capric acid, DHA, EPA, etc.), phospholipids (e.g. dipalmitoyl phophatidyl choline, soybean lecithin, phosphatidylcholine, etc.), chelating agents (e.g. ethylene-diamine-tetra-acetic acid (EDTA), citric acid, sodium citrate, sodium salicylate, etc.) and glycols (e.g. n-glycofurols, n-ethylene glycols, propylene glycol, isopropyl myristate, etc.).

Preservatives or Other Properties. Our experimental work suggests that preservatives could be important to maintaining the stability of the composition. As such, in some embodiments, the composition further comprises a preservative, such as benzalkonium chloride, potassium sorbate, phenylethyl alcohol (also known as 2-phenylethanol), 2-phenoxyethanol, or sodium azide.

The antiviral composition could be formulated to avoid or reduce irritation to the nasal passageways. This could be done by making the osmolarity of the composition close to isotonic (290 mOsm/L). In some embodiments, osmolarity in the range of 260-325 mOsm. Sodium chloride could be added to adjust the composition to the desired osmolarity. In some embodiments, the antiviral composition has a viscosity of 15-120 centipoise; and in some cases, 20-90 centipoise.

INTRANASAL DELIVERY DEVICE: In another aspect, the invention is an intranasal antiviral product, which may be a single-use or a multi-use product. The intranasal antiviral composition described herein is contained in an intranasal delivery device. Examples of intranasal delivery devices that could be used include nasal sprayers, pipettes, squeeze bottles, or squirt tubes. In some embodiments, the product is a multi-use product and the intranasal delivery device contains 3-20 ml volume of the antiviral composition. In some embodiments, the product is a single-use product and the intranasal delivery device contains less than 1.5 ml volume of the antiviral composition.

As explained below, it may be desirable to have local action in the nasal cavity or nasopharynx only and avoid deposition into the lungs or central airways (trachea and main-stem bronchi). As such, in some embodiments, the intranasal delivery device omits any propellants and is not pressurized. The topical intranasal composition of this invention is designed to form a viscous layer within the nasal passage that traps the virus and prevents it advancing further into the airway. Thus, limiting deposition to the nasal passages may be useful to avoid product waste and unwanted side effects. This can be done by generating droplet sizes that would deposit within the nasal passages, but not into the lungs. If the droplets are too small (e.g. <10 μm), they may pass through the nasal passages and deposit in the lungs instead. In some embodiments, the invention is not an inhalation spray device intended for drug delivery to the lungs. In some embodiments, the intranasal delivery device delivers liquid particles, wherein the particle size distribution is less than 20% of the particles having a diameter of <10 μm. More details about topical drug administration into the nasal cavity is described in Frank et al, “Effects of Anatomy and Particle Size on Nasal Sprays and Nebulizers” (2011) Otolaryngol Head Neck Surg. 146(2):313-319.

Any suitable amount of the antigen binder may be released with single actuation of the intranasal delivery device. In some embodiments, each actuation of the intranasal delivery device releases 30-175 μl volume of the antiviral composition. In some embodiments, each actuation of the intranasal delivery device releases 0.25 mg-4 mg of the antigen binder. In cases where multiple (two or more) types of antigen binders are being used (such as cocktails thereof or polyclonal antibodies), the amount refers to the total amount of all the different antigen binders in aggregate.

METHOD OF PROTECTION: In another aspect, this invention is a method of protecting against virus infection. The method comprises having an intranasal delivery device described herein. In some cases, the composition is released (e.g. by spraying or instilling droplets) into one or both nostrils such that the composition is deposited onto the mucosa of the nasal cavity or nasopharynx. It may be desirable to have local action in the nasal cavity or nasopharynx only. As such, in some embodiments, the composition is directly deposited onto the mucosa of the nasal cavity or nasopharynx only. Avoiding systemic absorption into the blood circulation may be desirable. As such, in some embodiments, the composition is not absorbed into the blood circulation. Also, avoiding the lungs or central airways (trachea and main-stem bronchi) may be desirable. As such, in some embodiments, the composition is not directly deposited into the lungs or central airways by the delivery device.

The intranasal delivery action (e.g. spray or droplets) may deliver any therapeutically effective amount of the antigen binder. In some embodiments, for each single actuation, the amount of the antigen binder released into the nasal cavity is 0.25 mg-4 mg. In cases where multiple types of antigen binders are being used (such as cocktails thereof or polyclonal antibodies), this amount refers to the total amount of all the different antigen binders in aggregate. Multiple actuations may be needed to achieve the full therapeutically effective amount of the antigen binder.

METHOD OF MANUFACTURE: In another aspect, this invention is a method of making the topical intranasal antiviral composition. The method comprises having a preparation of antigen binder. The antigen binder may be produced in any suitable manner, such as the techniques described herein. The antigen binder preparation may be in liquid or solid form. In liquid form, any suitable concentration amount of the antigen binder may be used. For example, the antigen binder preparation may be a liquid containing the antigen binder at a concentration of 5-150 mg/ml.

Separately from the antigen binder preparation, a pharmaceutical vehicle for the antigen binder is made. The pharmaceutical vehicle is made by making a homogenous aqueous mixture containing the pharmaceutical suspender. This homogenous aqueous mixture may be made by adding the pharmaceutical suspender to water or other aqueous solution. The mixture maybe homogenized by any appropriate agitation technique (e.g. stirring, shaking, etc.).

In embodiments where the pharmaceutical suspender is a combination of microcrystalline cellulose and sodium carboxymethylcellulose, any suitable amounts thereof may be used. In some embodiments, during the manufacturing process, the amount of microcrystalline cellulose used relative to the amount of sodium carboxymethylcellulose (by weight) is in the range of 20:1 to 3:1 (MCC:Na⋅CMC); in some cases, in the range of 15:1 to 5:1; and in some cases, in the range of 12:1 to 7:1. For example, the production method may use 9 times more microcrystalline cellulose than the amount of sodium carboxymethylcellulose (by weight).

In some embodiments, during the manufacturing process, the pharmaceutical suspender is in powder (solid) form for making the aqueous mixture. In some cases, the pharmaceutical suspender is provided as a powder blend of two or more different suspending agents. In some cases, the pharmaceutical suspender is a powder blend of microcrystalline cellulose and sodium carboxymethylcellulose. Any suitable ratio of the two suspending agents may be used. In some cases, the powder blend consists of microcrystalline cellulose and sodium carboxymethylcellulose in the following ratio (relative to the total weight of the powder blend representing 100%): 4-20 wt % sodium carboxymethylcellulose and 75-95 wt % microcrystalline cellulose.

The pharmaceutical suspender material, as used during the manufacturing process, could be characterized by its viscosity. In some embodiments, the pharmaceutical suspender has a viscosity of 20-200 centipoise (as tested when activated by mixing in a homogenizer with water at a concentration of 1.2 w/v % for 30 seconds duration); and in some cases, in the range of 30-115 centipoise. In cases where the pharmaceutical suspender comprises a blend of multiple (two or more) different suspending agents, this viscosity refers to the aggregate blend.

The pharmaceutical vehicle could be heat sterilized separate from the antigen binder preparation. This heat sterilization could be performed in any suitable manner. For example, the pharmaceutical vehicle could be autoclaved at a temperature of at least 115° C. for a duration of at least 20 minutes. The pharmaceutical vehicle is then cooled to an appropriate temperature (e.g. room temperature). After cooling, the antigen binder is added to the pharmaceutical vehicle to make a bioactive mixture. The bioactive mixture is made homogenous by any appropriate agitation technique (e.g. stirring, shaking, etc.).

Sodium chloride in solid form (as opposed to aqueous saline) is added to make the antiviral composition. For example, the solid sodium chloride could be powder, granules, particles, or other easily dispensable form. In the manufacturing process, the sodium chloride could be added to the bioactive mixture after it is made. Any suitable amount of sodium chloride may be added to reduce potential irritation in the nasal cavity. For example, the added sodium chloride may result in a topical antiviral composition containing 0.9 w/v % sodium chloride for isotonicity with the nasal cavity. In some embodiments, the pharmaceutical vehicle to which the sodium chloride is added has a sodium chloride concentration of less than 0.5 w/v %. In some embodiments, the sodium chloride is added at any time point after the pharmaceutical suspender, the antigen binder, or both.

Any use of the word “or” herein is intended to be inclusive and is equivalent to the expression “and/or,” unless the context clearly dictates otherwise. As such, for example, the expression “A or B” means A, or B, or both A and B. Similarly, for example, the expression “A, B, or C” means A, or B, or C, or any combination thereof.

### BRIEF DESCRIPTION OF THE DRAWINGS

FIG. 1 depicts an user spraying the antiviral composition into her nose.

FIG. 2 depicts a sagittal cross-section of the nasal cavity.

### DETAILED DESCRIPTION OF EXAMPLE EMBODIMENTS

To assist in understanding the invention, reference is made to experimental examples to show specific embodiments in which the invention may be practiced. Experimental work was performed to make a nasal spray composition using anti-S1 subunit IgY antibodies. Polyclonal IgY antibodies can be obtained from antigen-vaccinated egg-laying hens. The eggs are collected and the IgY antibodies are extracted from the egg yolk. This process is described in the literature, such as Amro et al, “Production and purification of IgY antibodies from chicken egg yolk” (2018) Journal of Genetic Engineering & Biotechnology, 16(1):99-103. This article is incorporated by reference herein.

In this work, egg-laying hens were immunized with the SARS-CoV-2 viral spike protein to induce IgY antibody production. The eggs were collected and anti-S1 subunit IgY antibodies were extracted from the egg yolks. Three different formulations were made at different concentrations of the IgY antibody: 5 mg/ml, 10 mg/ml, and 20 mg/ml. Described below is the process for making the 10 mg/ml formulation.

Preparing sterile antibody. The anti-S1 subunit IgY antibodies were provided at a target stock concentration 40 mg/ml. This was diluted to 24.7% (v/v) with a density of 1.066 g/ml. A 266 gram batch of the diluted antibody was filtered through a sterile syringe filter (which was a 0.2 μm cellulose acetate membrane) into a tared beaker.

Preparing pharmaceutical vehicle. A top entry mixer was set with the mixing shaft and impeller centered in a beaker and positioned close to the bottom of the beaker without touching. Sterile water (USP) was added to the beaker. The mixer was started from speed zero and the speed was increased slowly to stir the water to form a vortex without drawing air into the liquid. The target mixer speed was 2,000 rpm, but was adjusted as needed.

While continually stirring, 20 grams of Vivapur® MCG 591 P (2% w/v) was added to the beaker and mixed for about 10 minutes until an aqueous suspension was formed. Vivapur® MCG 591 P is a powder blend of microcrystalline cellulose (86.2-91.7 wt %) and sodium carboxymethylcellulose (8.3-13.8 wt %). After initial “overhead” mixing, the suspension was homogenized with stirring speed of about 5,000 rpm for a duration of about 30 minutes until the suspension turned into a homogenous gel. The beaker was then covered with aluminum foil and placed in the autoclave at a temperature of about 120° C. for about 30 minutes duration to sterilize the gel.

After autoclaving, resume stirring with the mixer while allowing the gel to cool to room temperature (about 25° C.). Continue stirring at 500 rpm for about 10 minutes while adding more sterile water (USP) to make the gel composition at the desired concentration of ingredients.

Adding the antibody. While continually stirring further at 700 rpm for about 10 minutes, add the sterile IgY antibody preparation (as described above) to the gel. Then while continually mixing further for about 5 minutes, add 9 grams of granular sodium chloride (USP, solid form) to make the gel 0.9% w/v isotonic saline.

This yielded a sterile gel composition with 10 mg/ml concentration of the IgY antibody in 0.9% saline, with microcrystalline cellulose and sodium carboxymethylcellulose at 2 w/v % in aggregate. The gel composition was dispensed into 3 ml size nasal spray vials with 1.5 ml of the gel composition in each vial.

Additional formulations. Similar compositions at 5 mg/ml and 20 mg/ml of the IgY antibody were made by dilution of the 40 mg/ml stock to about 12.5% and about 50% (w/v), respectively.

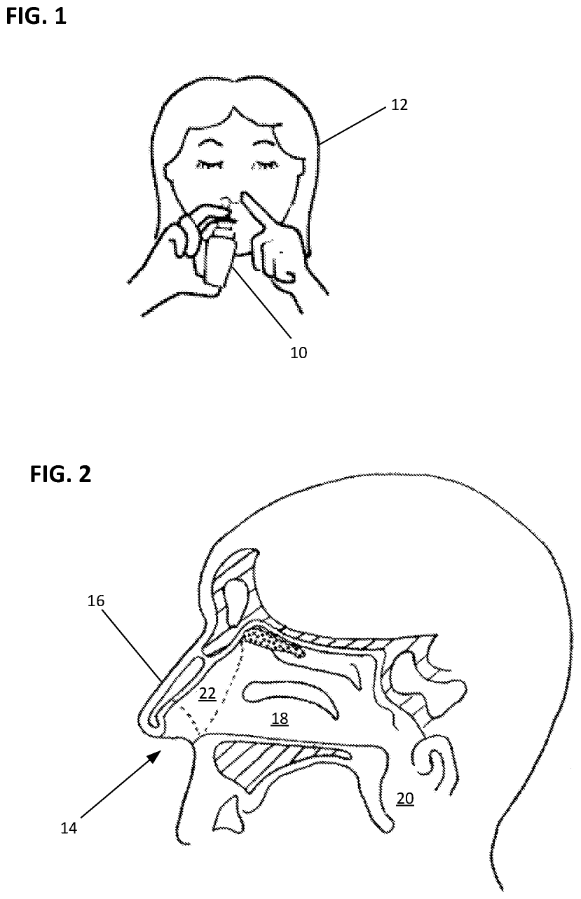

Drawing Figures. FIG. 1 depicts an example of how this invention could be used. Shown here is a user 12 having a nasal sprayer 10, which contains an antiviral composition of this invention. She is spraying the antiviral composition into her nose. FIG. 2 depicts an example of how nasal spray droplets could be limited to deposition in the nasal cavity. Shown here is the nose 16 and a sagittal cross-section of the nasal cavity with its vestibule 14, atrium 22, internal turbinates 18, and nasopharynx 20. Deposition of the nasal spray droplets may be limited to the nasal cavity without traveling further down into the lungs.

### Experimental Testing

The topical intranasal composition of this invention is designed to form a viscous layer within the nasal passage that traps the virus and prevents it advancing further into the airway. In order to achieve this, the composition must tolerate the high shear forces that are generated by the spray nozzle of nasal spray pumps. Shear forces may be problematic because they reduce the desired viscosity of the spray liquid. For experimental testing, we focused on blends of microcrystalline cellulose (MCC) and sodium carboxymethylcellulose (Na⋅CMC) for use as a pharmaceutical suspender. There are various grades of the product with different amounts of MCC and Na⋅CMC. Our experiments sought to find the mixture that was best suited for nasal spray of large biologic molecules, such as antibodies.

### Making the Pharmaceutical Vehicle

An initial study was conducted to select the suspending agent for making a suitable pharmaceutical vehicle to serve as a carrier for the IgY antibodies. Because the objective of the composition is to trap the virus with antibodies, thereby preventing entry of the virus into mucosal cells, the viscous nature of the composition is important for therapeutic efficacy. Prolonging residence time in the nasal cavity would enhance therapeutic efficacy.

To achieve this, a blend of MCC and Na⋅CMC was used to create the suspension. Table 1 below gives the different blend options that were tested. Viscosity testing was performed on a rotational viscometer. For initial setup, the viscometer was tested in plain water at room temperature to give the following results: viscosity of 11.72 centipoise (cPs) at 200 rpm speed, giving a torque of 62.4%, with accuracy of ±0.30 cPs. The following commercial products were tested for viscosity at a concentration of 1.5 w/v % in water. The viscosity results were as follows:

TABLE 1
Materials & Viscosity Testing

| Product | | Viscosity |
|---|---|---|
| Name | Materials | (cPs) |
| Avicel | microcrystalline cellulose and | 18.60 |
| CL-611 | Na · CMC (11.3-18.8 wt %) | |
| Avicel | microcrystalline cellulose and | 131.3 |
| RC-591 | Na · CMC (8.3-13.8 wt %) | |
| Vivapur | microcrystalline cellulose and | 99.70 |
| MCG 591P | Na · CMC (8.3-13.8 wt %) | |
| Methocel E5 | microcrystalline cellulose | Not tested |

After evaluating the results, we decided that Vivapur MCG 591P was most suitable based on the viscosity results. Also, visual inspection showed that the Vivapur behaved like a gel when undisturbed and transformed to a viscous liquid when shaken.

Because the IgY antibodies were provided in watery-thin aqueous form, it must be added to a proportionally thicker liquid to obtain a 1.5 wt % pharmaceutical vehicle. Thus, the Vivapur suspension was made in purified water at a concentration thicker than 1.5 wt %. An appropriate amount of IgY antibody was pipetted into each 25 ml volumetric flask and diluted to volume with the thicker Vivapur suspension. With this, the final mixture contained 1.5 wt % Vivapur at each concentration of the antibody. Table 2 below shows the proportions used for each strength.

TABLE 2
Proportions of Vivapur Relative to IgY

| | Control (0) | 5 mg/mL | | 10 mg/mL | | 20 mg/mL | |
|---|---|---|---|---|---|---|---|
| Vivapur | 1.5% | 1.7% | | 2.0% | | 3.0% | |
| Concentration | | | | | | | |
| IgY | N/A | 40 | mg/mL | 40 | mg/mL | 40 | mg/mL |
| Concentration | | | | | | | |
| Amount of IgY | N/A | 3 | mL | 6 | mL | 12.5 | mL |
| q.s to volume | N/A | 25 | mL | 25 | mL | 25 | mL |

We performed HPLC (high-performance liquid chromatograph) on the samples for quality control testing (see more details below). Table 3 below shows the proportions used for each antibody strength, along with the recovery amount of each sample. These HPLC recovery results confirmed that the technique of adding the IgY antibody to a proportionally thicker Vivapur suspension produced an acceptable formulation.

TABLE 3
IgY Recovery by HPLC

| | Control (0) | 5 mg/mL | | 10 mg/mL | | 20 mg/mL | |
|---|---|---|---|---|---|---|---|
| Vivapur | N/A | 1.7% | | 2.0% | | 3.0% | |
| Concentration | | | | | | | |
| IgY | N/A | 40 | mg/mL | 40 | mg/mL | 40 | mg/mL |
| Concentration | | | | | | | |
| Amount of IgY | N/A | 6 | mL | 12.5 | mL | 25 | mL |
| q.s. to volume | N/A | 50 | mL | 50 | mL | 50 | mL |

| | | | | |
|---|---|---|---|---|
| Recovery by | N/A | 94.1% | 95.8% | 98.1% |
| HPLC | | | | |

We then sought to make the pharmaceutical vehicle isotonic. Having the product be isotonic may reduce irritation to the nasal mucosa by avoiding inducement of osmotic flows. To create an isotonic suspension, sodium chloride was added to the homogenized Vivapur suspension. Sodium chloride 0.9% is a common fluid solution used in medical applications. To mimic this, we created a Vivapur suspension (without IgY) with 0.9% sodium chloride and studied the osmolality of the mixture. Table 4 below shows the osmolality of the resulting mixture (the target osmolality being 290 mOsm). These results indicate that a 1.5 wt % Vivapur suspension in 0.9% sodium chloride yielded the desired osmolality.

TABLE 4
Osmolality of Suspension with 0.9% Sodium Chloride (without IgY)

| | Osmolality (mOsm) |
|---|---|
| Trial 1 | 285 |
| Trial 2 | 287 |
| Trial 3 | 289 |
| Average | 287 |

We then studied how the IgY antibody alone (without saline) affected the solution osmolality. The testing results are shown in Table 5 below. These results indicate that the IgY antibody, at the different concentrations, has only minimal effect on overall osmolality. We believe that this minimal amount of solution osmolality is caused by the IgY antibody stock being provided in sodium acetate salt solution (as part of the protein reconstitution process performed by the supplier). Thus, we decided that the formulation could be made using 0.9% sodium chloride without the need to adjust the sodium chloride concentration according to the amount of IgY antibody. That is, the different strength antibody products could be made using the same sodium chloride addition technique.

TABLE 5
Osmolality (mOsm) without Sodium Chloride

| | 5 mg/ml | 10 mg/ml | 20 mg/ml |
|---|---|---|---|
| Trial 1 | 6 | 9 | 17 |
| Trial 2 | 5 | 9 | 16 |
| Trial 3 | 5 | 9 | 17 |
| Average | 5 | 9 | 17 |

We next performed visual observation of the formulation. This was done by dropping the liquid mixture onto a glass slide and making visual observations. Despite having satisfactory results on viscometer, HPLC, and osmolality, our visual observation indicated that more viscosity was needed. Thus, we decided to increase the Vivapur concentration to 2.0 wt % (up from 1.5 wt %). A comparison of the 1.5% versus the 2.0% formulation is shown in Table 6 below, using the 20 mg/mL strength for the IgY antibody, as an example. The IgY was calculated according to a presumed stock concentration of 40 mg/mL and density 1.066 g/ml. The q.s. was to 100 g of final suspension.

TABLE 6
Vivapur Amount Increased

| Raw Material | Formulation #1 (1.5%) | Formulation #2 (2.0%) |
|---|---|---|
| Vivapur MCG 591P | 1.5 g | 2.0 g |
| IgY Protein | 53.3 g | 53.3 g |
| Sodium Chloride | 0.9 g | 0.9 g |
| Purified Water | 35.0 g | 35.0 g |
| Purified Water | q.s. | q.s. |

Osmolality testing was conducted on Formulation #2 above to observe how increasing the concentration of Vivapur affected the osmolality. Table 7 below shows the results. As seen here, the osmolarity increased slightly compared to the about 290 mOsm expected for the 0.9% sodium chloride solution alone. This is because the IgY protein stock was provided in sodium acetate solution, which contributed slightly to the osmolality. This slight increase in osmolality is acceptable. We also measured density to modify the amount of added purified water at the end of the manufacturing process. The measured density of Formulation #2 was 1.077 g/mL and the acidity was pH 6.75.

TABLE 7
Osmolality Testing

| Trial | Osmolality (mOsm) |
|---|---|
| Trial 1 | 308 |
| Trial 2 | 309 |
| Trial 3 | 313 |
| Average | 310 |

Benefits of Homogenization. Originally, the Vivapur and purified water were only mixed with a top mixer. Although this achieved acceptable results, we found that homogenizing the suspension at high shear forces made it thicker more without having to add additional Vivapur. Table 8 below shows Formulations #1-3. Formulation #3 gave the best viscosity and gelling properties. The IgY was calculated according to a presumed stock concentration of 40 mg/ml and density 1.066 g/ml. For #1 and #2, the q.s. was to 100 grams of final suspension. For #3, the q.s. was to 107.7 g of final suspension. The amounts are expressed as gram units.

TABLE 8
Effect of Homogenization

| Raw Material | Formulation #1 | Formulation #2 | Formulation #3 |
|---|---|---|---|
| Vivapur MCG 591P | 1.5 | 2.0 | 2.0 |
| IgY Protein | 53.3 | 53.3 | 53.3 |
| Sodium Chloride | 0.9 | 0.9 | 0.9 |
| Purified Water | 35.0 | 35.0 | 40.0 |
| Purified Water | q.s. | q.s. | q.s. |
| Homogenization | X | X | ✓ |

Tables 9-12 below show the final formulations for the control (no IgY), 5 mg/mL, 10 mg/mL, and 20 mg/mL strengths, respectively. Table 13 gives a summary of the cGMP (Current Good Manufacturing Practice) manufacturing process.

TABLE 9
Final Formulation, Placebo Control

| Ingredient | w/w % | Batch Weight (g) |
|---|---|---|
| Vivapur MCG 591P | 2.0 | 20.0 |
| Sodium Chloride | 0.9 | 9.0 |
| Sterile Water | q.s. | Portion 1: 533 |
| | | Portion 2: q.s |

| | |
|---|---|
| Total | 1077 |

TABLE 10
Final Formulation, IgY at 5 mg/mL

| Ingredient | w/w % | Batch Weight (g) |
|---|---|---|
| Vivapur MCG 591P | 2.0 | 20.0 |
| Anti-S1 Protein IgY | 12.4 | 133.5 |
| Sodium Chloride | 0.9 | 9.0 |
| Sterile Water | q.s. | Portion 1: 533 |
| | | Portion 2: q.s |

| | |
|---|---|
| Total | 1077 |

TABLE 11
Final Formulation, IgY at 10 mg/mL

| Ingredient | w/w % | Batch Weight (g) |
|---|---|---|
| Vivapur MCG 591P | 2.0 | 20.0 |
| Anti-S1 Protein IgY | 24.7 | 266.0 |
| Sodium Chloride | 0.9 | 9.0 |
| Sterile Water | q.s. | Portion 1: 533 |
| | | Portion 2: q.s |

| | |
|---|---|
| Total | 1077 |

TABLE 12
Final Formulation, IgY at 20 mg/mL

| Ingredient | w/w % | Batch Weight (g) |
|---|---|---|
| Vivapur MCG 591P | 2.0 | 20.0 |
| Anti-S1 Protein IgY | 49.4 | 533 |
| Sodium Chloride | 0.9 | 9.0 |
| Sterile Water | q.s. | Portion 1: 500 |
| | | Portion 2: q.s |

| | |
|---|---|
| Total | 1077 |

TABLE 13
Summary of cGMP Manufacturing Process

| Step # | Procedure |
|---|---|
| 1 | All product contact materials are sterilized prior to batch |
| | start to minimize potential bioburden. |
| 2 | A calculation is performed based upon the purity of the |
| | anti-S1 protein IgY to determine if any adjustments to the |
| | theoretical water quantity are required. |
| 3 | The anti-S1 protein IgY is dispensed first. As the anti-S1 |
| | protein IgY is dispensed it is passed through a 0.22 μm |
| | filter to remove any bioburden. |
| 4 | All other ingredients are dispensed. |
| 5 | Water is added to a suitable container and mixing begins |
| | using a top entry mixer to form a vortex without |
| | introduction of air. |
| 6 | Vivapur MCG 591P is then added and mixed for at least 10 |
| | minutes to ensure homogeneity. |
| 7 | This suspension is then homogenized using high shear |
| | mixing for at least 30 minutes to form a more viscous |
| | suspension. |
| 8 | This suspension is then autoclaved to for at least 30 minutes |
| | at a minimum temperature of 120° C. to remove |
| | any bioburden. |
| 9 | The suspension is then cooled down to ≤25° C. |
| 10 | The resultant suspension is then weighed, and additional |
| | water is dispensed and added to make up for any evaporation |
| | losses during sterilization, etc., that have occurred to this |
| | point. The suspension is then mixed for at least 10 |
| | minutes to ensure homogeneity. |
| 11 | The batch yield is then calculated to ensure there are no |
| | irregularities prior to adding the anti-S1 protein IgY. |
| 12 | The anti-S1 protein IgY is then added and mixed at a slower |
| | speed for at least 10 minutes. |
| 13 | The sodium chloride is then added for isotonicity |
| | and then mixed for at least 20 minutes to ensure |
| | homogeneity. |
| 14 | Additional steps are then performed to calculate final |
| | yield and remove the appropriate samples. |
| 15 | The suspension is then packaged by adding 1.5 mL of |
| | suspension to a 3 mL dropper bottle w/control tip and |
| | placing the cap on the bottle. This process continues until |
| | the desired quantity of bottles have been filled. |

Technical Observations: Sodium chloride was added to the mixture to make the suspension liquid isotonic. Having an isotonic liquid would avoid or reduce irritation to the nasal passageways. In the step where sodium chloride granules were added to the mixture, we observed that adding the sodium chloride after doing the high-temperature autoclaving was important. When the sodium chloride was added to the pharmaceutical vehicle initially and then autoclaved at high temperature, this caused the Vivapur blend to precipitate out of the mixture. This is an undesirable effect. Thus, adding sodium chloride in solid form (e.g. powder or granules) to the liquid composition after the IgY or the Vivapur may be critical to making a workable product. This is as opposed to adding IgY or Vivapur to an already-prepared saline sodium chloride aqueous solution.

Also, performing the high-temperature autoclave sterilization of the pharmaceutical vehicle before adding the IgY antibodies avoids the possibility of causing the antibodies to denature or degrade. Thus, pharmaceutical vehicle was autoclaved prior to adding the sodium chloride and separately from the sterilizing the antibody preparation.

In adding the IgY antibody to the pharmaceutical vehicle, we observed that the mixing should be performed slowly. Otherwise, it creates a frothy liquid instead of a gel. We believe that this is because the IgY antibody is an egg-based protein. Also, for storage, freezing the composition may be undesirable because the Vivapur excipients may separate out from the mixture during thawing.

### Stability Testing

The following stability testing was performed at both refrigerated (2-4° C.) and room temperature. The duration of the stability testing was three months, and in some batches, up to six months.

pH Stability: The pH of the IgY stock was close to physiological. However, because the suspension formulation did not contain any buffers, unwanted changes in pH was a possibility. Thus, we tested for stability of pH and the results are shown in Tables 14 & 15. These results indicate that the pH of the suspension is stable in both temperature conditions.

TABLE 14
pH stability; refrigerated

| Batch | Initial | 2 wk. | 1 mon. | 2 mon. | 3 mon. | 6 mon. |
|---|---|---|---|---|---|---|
| #1-Control (0) | 5.6 | 5.6 | 5.7 | 5.6 | 5.7 | N/A |
| #2-5 mg/ml | 6.5 | 6.4 | 6.5 | 6.5 | 6.5 | |
| #3-10 mg/ml | 6.7 | 6.7 | 6.6 | 6.7 | 6.7 | |
| #4-20 mg/ml | 6.8 | 6.8 | 6.7 | 6.8 | 6.8 | 6.1 |

TABLE 15
pH stability; room temperature

| Batch | initial | 2 wk. | 1 mon. | 2 mon. | 3 mon. |
|---|---|---|---|---|---|
| #1 - Control (0) | 5.6 | 5.6 | 5.6 | 5.6 | 5.7 |
| #2 - 5 mg/ml | 6.5 | 6.5 | 6.5 | 6.5 | 6.5 |
| #3 - 10 mg/ml | 6.7 | 6.7 | 6.6 | 6.7 | 6.7 |
| #4 - 20 mg/ml | 6.8 | 6.8 | 6.8 | 6.8 | 6.7 |

Osmolality Stability: One of the objectives of the formulation design was isotonicity to avoid or reduce irritation to the nasal passageways. In the suspension, there are two factors affecting osmolality: sodium chloride concentration and IgY stock concentration. Because the IgY stock contained a small amount of sodium acetate, this could potentially affect the desired isotonic osmolality. Thus, we tested for stability of osmolality and the results are shown in Tables 16 & 17. These results indicate that the osmolality of the suspension is stable in both temperature conditions.

TABLE 16
Osmolality stability (mOsm); refrigerated

| Batch | Initial | 2 wk. | 1 mon. | 2 mon. | 3 mon. | 6 mon. |
|---|---|---|---|---|---|---|
| #1-Control (0) | 275 | 277 | 277 | 274 | 273 | N/A |
| #2-5 mg/ml | 358 | 355 | 311 | 230 | 326 | |
| #3-10 mg/ml | 288 | 284 | 286 | 289 | 288 | |
| #4-20 mg/ml | 295 | 294 | 293 | 293 | 295 | 295 |

TABLE 17
Osmolality stability (mOsm); room temperature

| Batch | Initial | 2 wk. | 1 mon. | 2 mon. | 3 mon. |
|---|---|---|---|---|---|
| #1 - Control (0) | 275 | 279 | 274 | 274 | 275 |
| #2 - 5 mg/ml | 358 | 233 | 241 | 320 | 310 |
| #3 - 10 mg/ml | 288 | 287 | 288 | 290 | 287 |
| #4 - 20 mg/ml | 295 | 295 | 292 | 292 | 311 |

For #2, the variance in osmolality was caused by inaccurate measurements from mixing problems.

Viscosity Stability: The viscosity of nasal spray compositions are known to change over time. As mentioned above, having sufficient viscosity is an important factor to the topical nasal composition. Thus, we tested for stability of viscosity and the results are shown in Tables 18 & 19. Viscosity here is measured as centipoise (cPs). These results indicate that the viscosity of the suspension is stable in both temperature conditions.

TABLE 18
Viscosity stability (cPs), refrigerated

| Batch | Initial | 2 wk. | 1 mon. | 2 mon. | 3 mon. |
|---|---|---|---|---|---|
| #1 - Contral (0) | 48 | 45 | 48 | 48 | 50 |
| #2 - 5 mg/ml | 52 | 63 | 59 | 52 | 57 |
| #3 - 10 mg/ml | 51 | 56 | 55 | 57 | 60 |
| #4 - 20 mg/ml | 47 | 51 | 54 | 53 | 52 |

TABLE 19
Viscosity stability (cPs), room temperature

| Batch | Initial | 2 wk. | 1 mon. | 2 mon. | 3 mon. |
|---|---|---|---|---|---|
| #1 - Control (0) | 48 | 43 | 50 | 51 | 51 |
| #2 - 5 mg/ml | 52 | 57 | 57 | 54 | 56 |
| #3 - 10 mg/ml | 51 | 63 | 53 | * | 61 |
| #4 - 20 mg/ml | 47 | 51 | 53 | 53 | 52 |

*Not performed.

IgY Stability: Being a biologic product, the IgY antibodies are vulnerable to degradation by a variety of factors. Thus, we developed an HPLC (high performance liquid chromatography) protocol to detect intact IgY for quality control purposes. In developing the HPLC protocol, we observed that slowing the flow rate from 0.5 to 0.4 ml/min enhanced the resolution between peaks for quantitative analysis. This HPLC protocol gave us the ability to accurately quantify the amount of intact IgY recovered. Tables 20 & 21 show the IgY amounts that were recovered.

TABLE 20
IgY stability (% recovery by HPLC), refrigerated

| Batch | Initial | 2 wk. | 1 mon. | 2 mon. | 3 mon. | 6 mon. |
|---|---|---|---|---|---|---|
| #1-Control (0) | N/A | N/A | N/A | N/A | N/A | N/A |
| #2-5 mg/ml | 96% | 96% | 97% | 95% | 99% | |
| #3-10 mg/ml | 97% | 99% | 98% | 98% | 99% | |
| #4-20 mg/ml | 96% | 98% | 96% | 96% | 100% | 102% |

TABLE 21
IgY stability (% recovery by HPLC), room temperature

| Batch | initial | 2 wk. | 1 mon. | 2 mon. | 3 mon. |
|---|---|---|---|---|---|
| #1 - Control (0) | N/A | NA | N/A | N/A | N/A |
| #2 - 5 mg/ml | 96% | 100% | 99% | 97% | 103% |
| #3 - 10 mg/ml | 97% | 100% | 99% | 96% | 104% |
| #4 - 20 mg/ml | 96% | 99% | 97% | 100% | 99% |

Visual Observations: We also performed visual inspections of the batches through the three month testing duration. There were no irregularities in the refrigerated batches for the three month duration. However, there were irregularities in the room temperature batches. In a few of the samples, there was a color change to pale yellow, appearance of dark-colored foreign substances, or strong sulfuric odor. This happened in the 5 mg/ml batch at the three-month timepoint and thereon; in the 10 mg/ml batch at the one-month time point and thereon; and in the 20 mg/ml batch at the one-month timepoint and thereon.

1. A method of making a topical intranasal antiviral composition, comprising:
having a preparation of antigen binder;
making a pharmaceutical vehicle comprising a pharmaceutical suspender;
heat sterilizing the pharmaceutical vehicle;
cooling the pharmaceutical vehicle;
adding the antigen binder to the pharmaceutical vehicle to make a bioactive mixture;
adding solid sodium chloride to the bioactive mixture.

2. The method of claim 1, wherein the antigen binder preparation is a liquid containing the antigen binder at a concentration of 5-150 mg/ml.

3. The method of claim 1, wherein the pharmaceutical vehicle comprises a homogenous aqueous mixture containing the pharmaceutical suspender.

4. The method of claim 3, wherein the homogenous aqueous mixture is made by adding the pharmaceutical suspender to water or an aqueous solution.

5. The method of claim 4, wherein the pharmaceutical suspender comprises microcrystalline cellulose and sodium carboxymethylcellulose.

6. The method of claim 5, wherein the pharmaceutical suspender is added to the water or aqueous solution as a powder blend consisting essentially of microcrystalline cellulose and sodium carboxymethylcellulose.

7. The method of claim 1, wherein the pharmaceutical suspender is a powder blend comprising microcrystalline cellulose and sodium carboxymethylcellulose.

8. The method of claim 7, wherein the powder blend is 4-20 wt % sodium carboxymethylcellulose and 75-95 wt % microcrystalline cellulose.

9. The method of claim 8, wherein the amount of microcrystalline cellulose in the blend relative to the amount of sodium carboxymethylcellulose (by weight) is in the range of 20:1 to 3:1 (MCC:Na⋅CMC).

10. The method of claim 9, wherein the antiviral composition comprises 0.04-0.70 wt % Na⋅CMC.

11. The method of claim 8, wherein the powder blend has a viscosity of 20-200 centipoise.

12. The method of claim 1, wherein the antigen binder is polyclonal IgY antibodies.

13. The method of claim 1, wherein the antigen binder is targeted to a coronavirus spike protein.

14. The method of claim 13, wherein the antigen binder is targeted to an S1 subunit of coronavirus spike protein.

15. The method of claim 13, wherein the coronavirus is SARS-CoV-2.

16. The method of claim 1, wherein the antiviral composition has a viscosity of 15-120 centipoise.

17. The method of claim 1, wherein the osmolarity of the antiviral composition is in the range of 260-325 mOsm.

18. The method of claim 1, further comprising adding a preservative.

19. The method of claim 1, wherein the sodium chloride is added after making the bioactive mixture.

20. The method of claim 4, wherein the step of adding the sodium chloride is performed at a time after adding the pharmaceutical suspender.
